(19) United States
(12) Patent Application Publication
(10) Pub. No.: US 2016/0263308 A1
(43) Pub. Date: Sep. 15, 2016

(54) ORGANIZATIONAL SYSTEM AND METHODS FOR MEDICATION SYRINGES

(71) Applicant: Anh L. Ngo, Las Vegas, NV (US)

(72) Inventor: Anh L. Ngo, Las Vegas, NV (US)

(21) Appl. No.: 15/076,448

(22) Filed: Mar. 21, 2016

Related U.S. Application Data
(63) Continuation-in-part of application No. 14/996,129, filed on Jan. 14, 2016.
(60) Provisional application No. 62/103,518, filed on Jan. 14, 2015.

(51) Int. Cl. A61M 5/00; A61B 50/30

(52) U.S. Cl. CPC A61M 5/002; A61B 50/30; A61M 2205/584; A61M 2209/00; A61M 2205/6081; A61B 2050/318; A61B 2050/3008

(57) ABSTRACT

Systems and methods are provided for syringe organization including a backing, a plurality of pockets coupled to the backing and a coordination scheme indicating the contents of individual pockets.

### CROSS-REFERENCE TO OTHER RELATED APPLICATIONS

The present application is a continuation-in-part of U.S. patent application Ser. No. 14/996,129 filed January 14, 2016 for “ORGANIZATIONAL SYSTEM FOR MEDICATION SYRINGES,” which claims priority to U.S. Provisional Application No. 62/103,518 filed Jan. 14, 2015 titled “ORGANIZATIONAL SYSTEM FOR MEDICATION SYRINGES,” both of which are hereby incorporated by reference in their entirety and for all purposes.

### FIELD

The subject matter described herein relates generally to a system, apparatus and method for organizing medication syringes.

### BACKGROUND

Incorrect medication administration in the operating room is a major patient safety issue worldwide. Numerous studies have been conducted on the subject and incidents may still be grossly underreported, often with devastating outcomes.

Anesthesia is one of the few areas in healthcare with no secondary verification of medication administration. Anesthesia also has the highest number of administered medications, most of which are high-alert medications. High alert medications are drugs that bear a heightened risk of causing significant patient harm when they are used in error. Anesthesiologists and associated anesthesia providers such as Nurse Anesthetists and Anesthesia Assistants are on the front line where medications are prescribed, mixed, relabeled and administered without safety checks or secondary verification. The process of mixing, preparing, and organizing medications as performed by individual anesthesia providers can be complicated, even in quiet environments. Often the process takes place in busy and stressful environments such as operating rooms, which can reduce a provider's attentiveness to the tasks performed and compound the likelihood of errors.

In the year 2000, the Institute of Medicine (IOM) declared a critical need to improve patient safety by learning from errors, raising standards, and creating systems that ensure the use of safe practices at the delivery level of healthcare. The TOM reported that a total of $29 billion is spent each year on preventable adverse events with $2 billion of this sum being attributed to adverse drug events (ADEs). The IOM also reported that medical errors injure at least 1.5 million Americans each year and cause 7,000 deaths annually.

According to a 2006 Institute of Medicine Report, “Preventing Medical Errors,” on average, a hospitalized patient is subject to at least 1 medication error per day, with at least 1.5 million preventable adverse drug reactions occurring each year. These reactions lead to an estimated $3.5 billion in additional health care costs annually to hospitalized patients alone, and adverse reactions to drugs represents the fourth leading cause of deaths in hospitalized patients.

According to an analysis of over 73,000 intravenous drug errors reported to the US Pharmacopoeia MedMarx database between 2000 and 2004, more than 50% of errors occurred during the process of administering medications and 60% of these errors occurred in the intravenous administration of 1 of 20 “high alert” medications. Between 3% and 5% of these reported errors led to patient harm.

Additional information can be found in “To Err is Human: Building a Safer Health System” ISBN 0-309-51563-7; “Preventing Medication Errors” ISBN 0-309-65856-X; and “Standardizing Medication Error Event Reporting in the U.S. Department of Defense” by Ronald A. Nosek, Jr. Other information can be found at

(http://store.usp.org/OA_HTML/ibeCCtpItmDspRte.jsp?item=876081&section=10071&beginIn dex=0&sitex=10020:22372:US) and
(http://www.apsforg/newsletters/html/2008/fall/01errors.htm)

Current solutions to this problem include placement of medication syringes on a countertop according to personal preference of the individual administering the medication. Syringe size, planned time of administration, preferred color code and other schemes exist. However, these schemes are ineffective because they can easily become cluttered, disorganized and otherwise inefficient, inaccurate and potentially dangerous. This situation can be further exacerbated if medication providers forget to label medication syringes or grab the wrong syringe due to urgent and stressful situations.

As such, improved methods and systems of medication syringe organization are desirable.

### SUMMARY

Provided herein are embodiments of systems and methods of organizing medication syringes hanging on an IV pole, surgical or operating bed, or affixed or laid out on an anesthesia machine, drug cart or other surface in an anesthetic or other procedural area. These systems and methods provide a standardized organization scheme for preoperative, intraoperative, and postoperative and intensive care unit medication syringe location by coded medication classifications. These classifications can be based off of the same medication classifications provided by pharmacies and drug manufacturers. In some embodiments, this can include classifications or standards published by the American Society for Testing and Materials (ASTM) standard for user-applied labels in the operating room (OR) (ASTM D4774-06 Standard Specification for User Applied Drug Labels in Anesthesiology, such as those listed at:

{http://www.astm.org/DATABASE.CART/HISTORICAL/D4774-06.htm}.

The systems and methods described herein provide improved recognition in the form of simplified coding in addition to full labeling recognition of medications commonly used before, during and after an operative course. Additionally, medication administrators can organize syringes filled with medications or syringes intended to be used with particular medications to be placed in appropriately labeled groups and subgroups or medication vials to be used with syringes.

The systems and methods described herein can be applied to procedural, surgical and operating suites including pediatric, orthopedic, spine, urology, otolaryngology, plastic surgery, neurosurgery, cardiovascular, obstetrics and gynecology, ophthalmology, general surgery, cosmetic surgery, battlefield surgery, trauma surgery, interventional radiology, interventional cardiology, interventional pain management, gastroenterology procedural, cosmetic procedural, outpatient, inpatient, labor and delivery procedural and operating, emergency room trauma, intensive care inpatient, dental, and other suites and rooms.

Other systems, devices, methods, features and advantages of the subject matter described herein will be or will become apparent to one with skill in the art upon examination of the following figures and detailed description. It is intended that all such additional systems, devices, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1A is an example view of a disorganized syringe table as known in the prior art.

FIG. 1B is an example view of a disorganized syringe table as known in the prior art.

FIG. 1C is an example view of a disorganized syringe table in an operation room as known in the prior art.

FIG. 1D is an example view of a disorganized syringe table in an operation room as known in the prior art.

FIG. 2 is an example embodiment of a medication syringe organization apparatus diagram in accordance with the present invention.

FIG. 3 is an example embodiment of a medication syringe organization apparatus from a front view in accordance with the present invention.

FIG. 4 is an example embodiment of a medication syringe organization apparatus from a front side perspective view in accordance with the present invention.

FIG. 5 is an example embodiment of a medication syringe organization apparatus from a front side perspective view in accordance with the present invention.

FIG. 6 is an example embodiment of a medication syringe organization apparatus from a back view in accordance with the present invention.

FIG. 7 is an example embodiment of a medication syringe organization apparatus from a back side perspective view in accordance with the present invention.

FIG. 8 is an example embodiment of a medication syringe organization apparatus header area in accordance with the present invention.

FIG. 9 is an example embodiment of a medication syringe organization apparatus header area in accordance with the present invention.

FIG. 10 is an example embodiment of a medication syringe organization apparatus pocket area first row in accordance with the present invention.

FIG. 11 is an example embodiment of a medication syringe organization apparatus pocket area second row in accordance with the present invention.

FIG. 12 is an example embodiment of a medication syringe organization apparatus pocket area third row in accordance with the present invention.

FIG. 13 is an example embodiment of a medication syringe organization apparatus pocket area fourth row in accordance with the present invention.

FIG. 14 is an example embodiment of a standardized label coloring chart for anesthetic medications in the United States in accordance with the present invention.

FIGS. 15A-B show an example embodiment of a first and second section respectively of a classification chart.

FIG. 16 is an example embodiment of a medication syringe organization apparatus diagram in accordance with the present invention.

FIG. 17A is an example embodiment of a medication syringe organization apparatus from a front side perspective view in accordance with the present invention.

FIG. 17B is an example embodiment of a medication syringe organization apparatus header area in accordance with the present invention.

FIG. 17C is an example embodiment of a medication syringe organization apparatus pocket area first row in accordance with the present invention.

FIG. 17D is an example embodiment of a medication syringe organization apparatus pocket area second row in accordance with the present invention.

FIG. 17E is an example embodiment of a medication syringe organization apparatus pocket area third row in accordance with the present invention.

FIG. 17F is an example embodiment of a medication syringe organization apparatus pocket area fourth row in accordance with the present invention.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a”, “an”, and “the” include plural referents unless the context clearly dictates otherwise.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

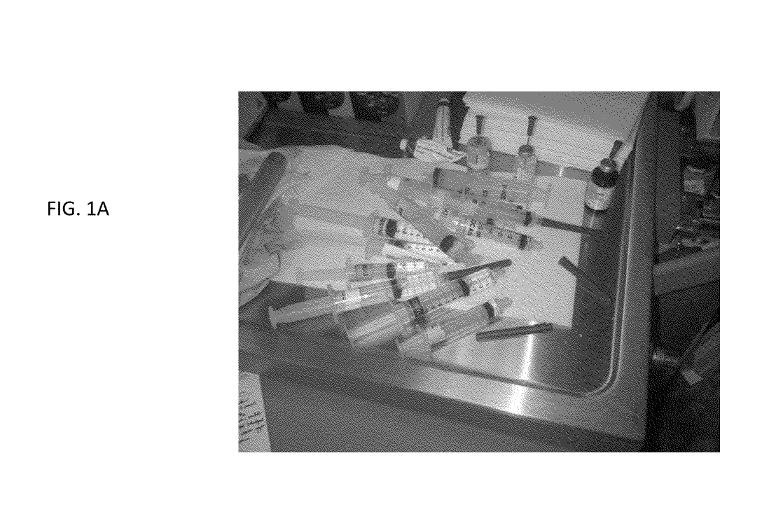

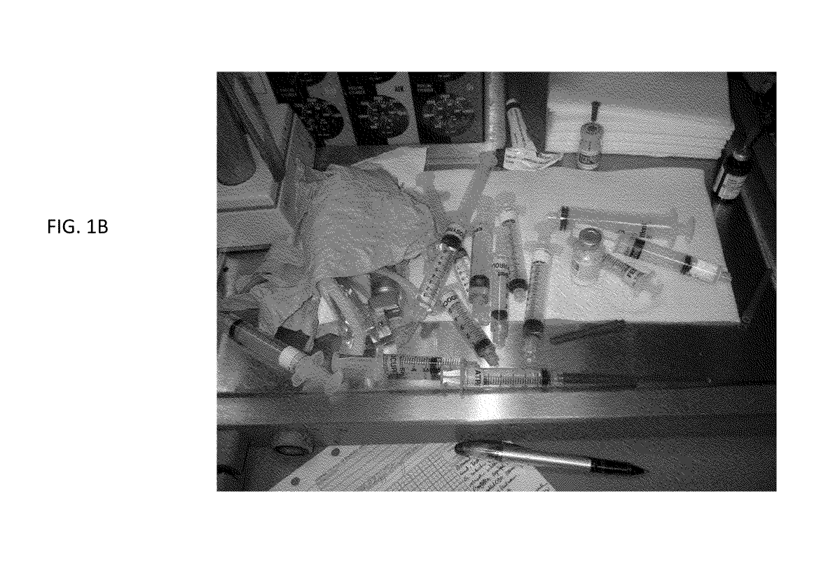

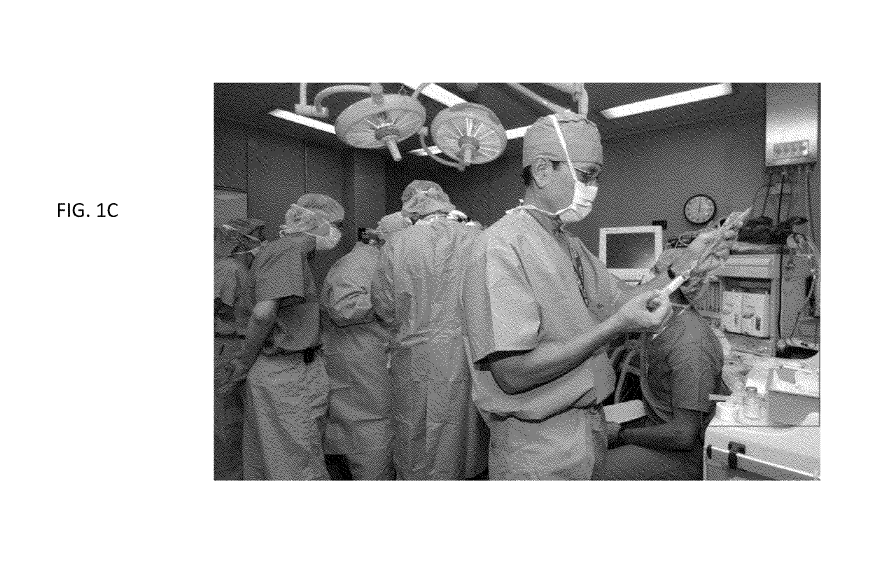

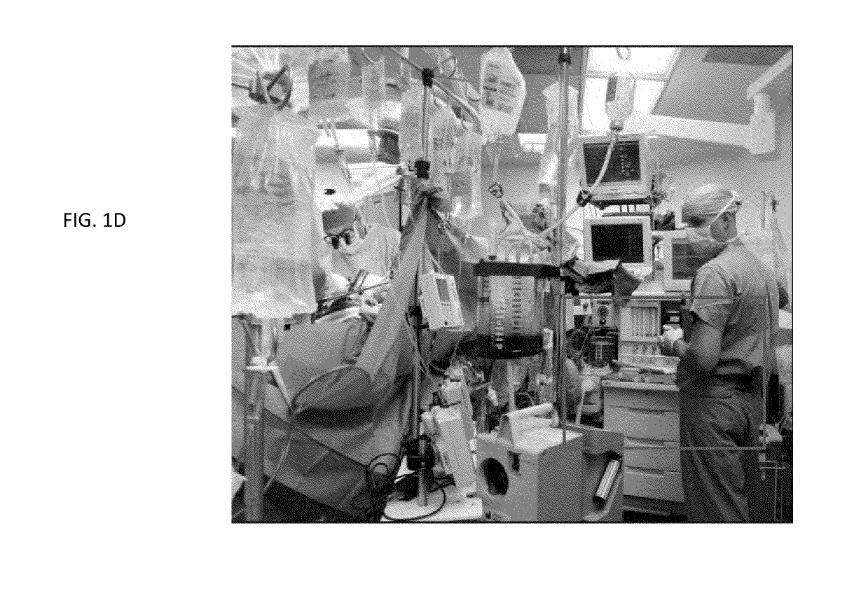

Turning to FIGS. 1A-1B, disorganized syringe tables as known in the prior art are shown. In the example embodiments syringes are strewn about the table. Some syringes are labeled while others are not. Some syringes have needles attached while others do not. Other implements are located on the table in a wholly disorganized and haphazard fashion.

Turning to FIGS. 1C-1D, example views of a disorganized syringe table in an operation room as known in the prior art are shown. In the example embodiments, busy and crowded operating rooms are shown in which medication syringe tables can be bumped, contacted and otherwise disorganized by any of the personnel in the room.

Many of the example embodiments described herein are based on standards created by the American Society for Testing and Materials (ASTM) International, in particular the ASTM D4774 standard: “Standard Specification for User Applied Drug Labels in Anesthesiology.” It should be understood that various other former, current and later developed standards can be implemented with and benefit in similar fashion from the concepts and embodiments described herein and thus, these concepts and embodiments are not limited to only the ASTM standards.

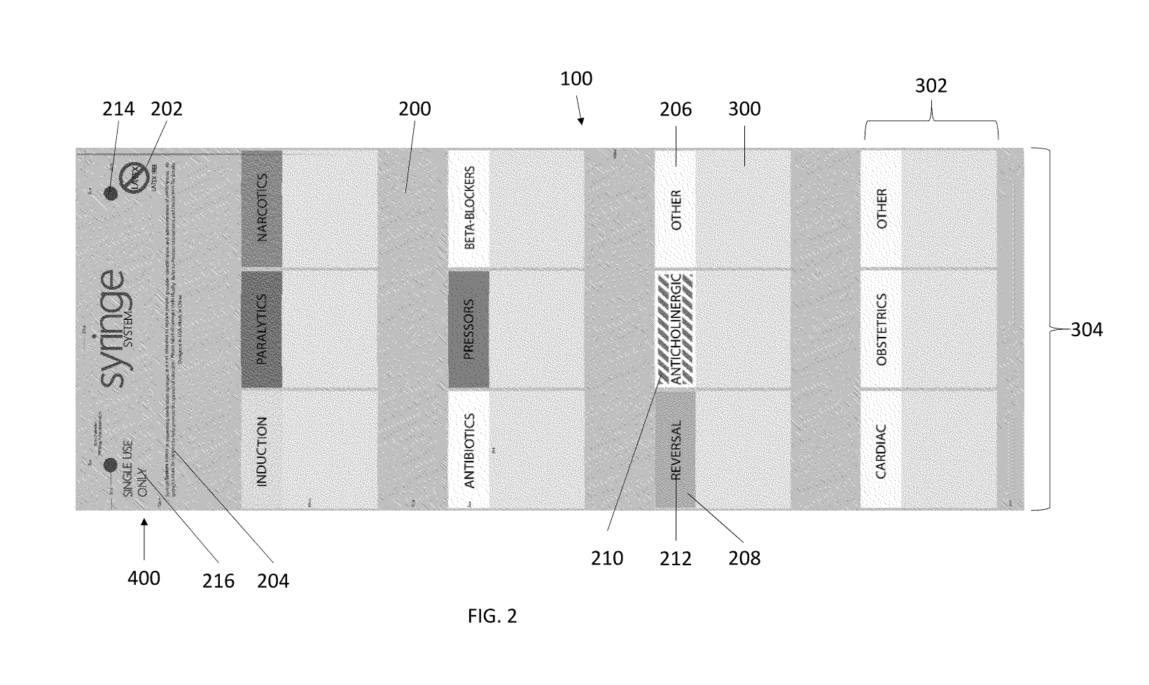

Turning to FIGS. 2-13, example embodiments of a medication syringe organization system and apparatus 100 are shown.

FIG. 2 is an example embodiment of a medication syringe organization apparatus diagram 100 in accordance with the present invention.

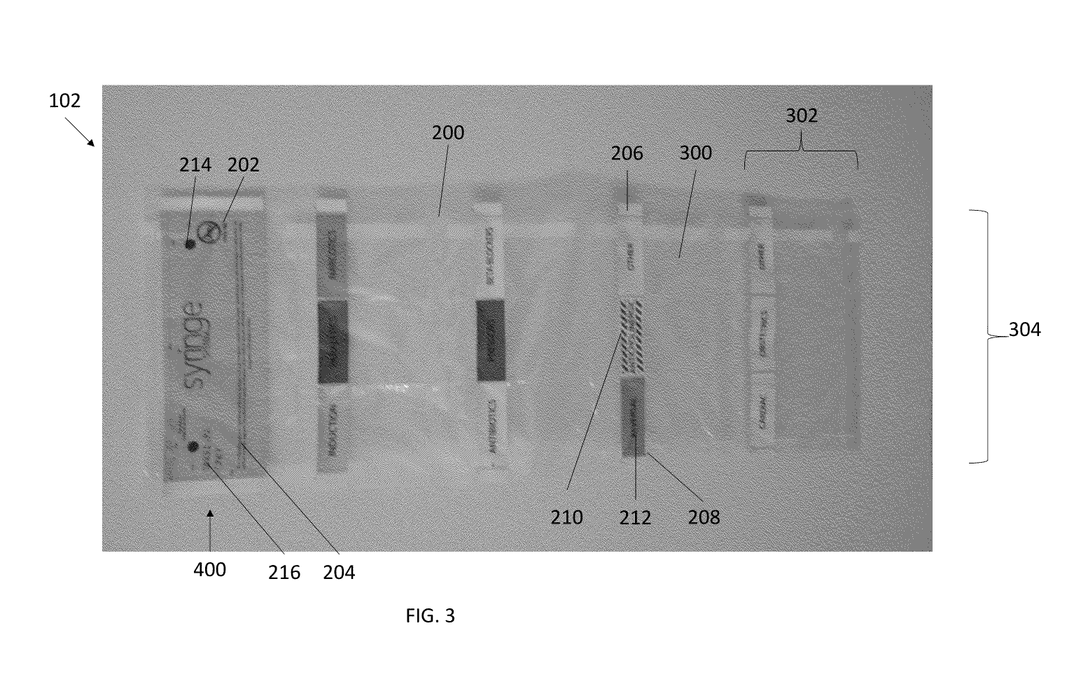

FIG. 3 is an example embodiment of a medication syringe organization apparatus 102 from a front view in accordance with the present invention.

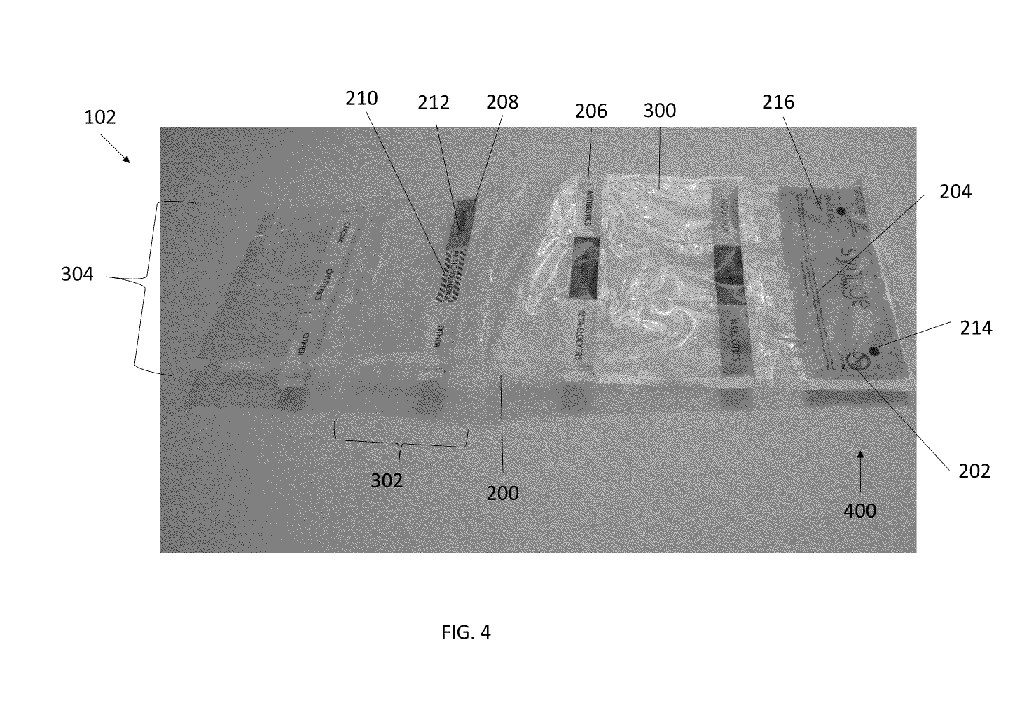

FIG. 4 is an example embodiment of a medication syringe organization apparatus 102 from a front side perspective view in accordance with the present invention.

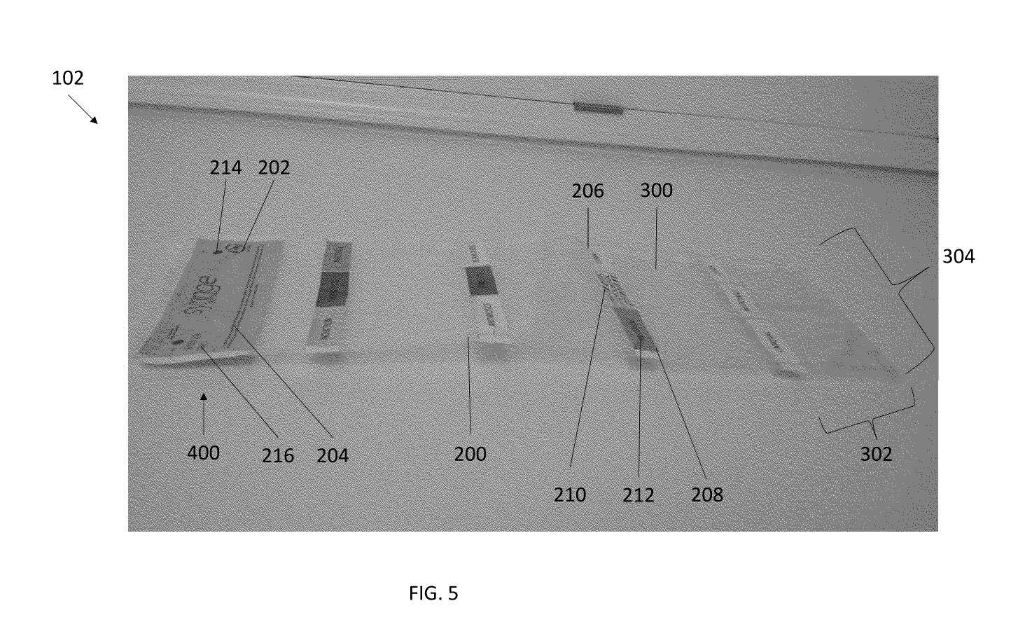

FIG. 5 is an example embodiment of a medication syringe organization apparatus 102 from a front side perspective view in accordance with the present invention.

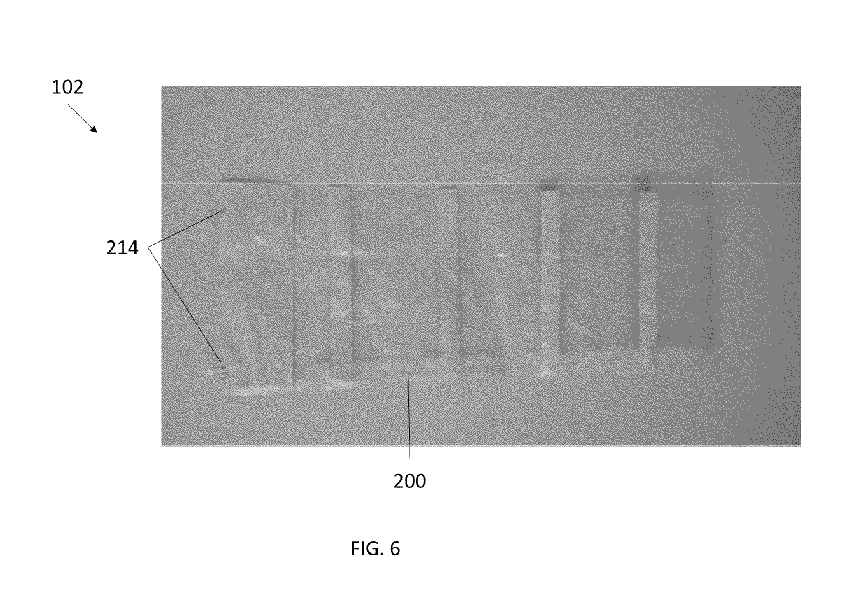

FIG. 6 is an example embodiment of a medication syringe organization apparatus 102 from a back view in accordance with the present invention.

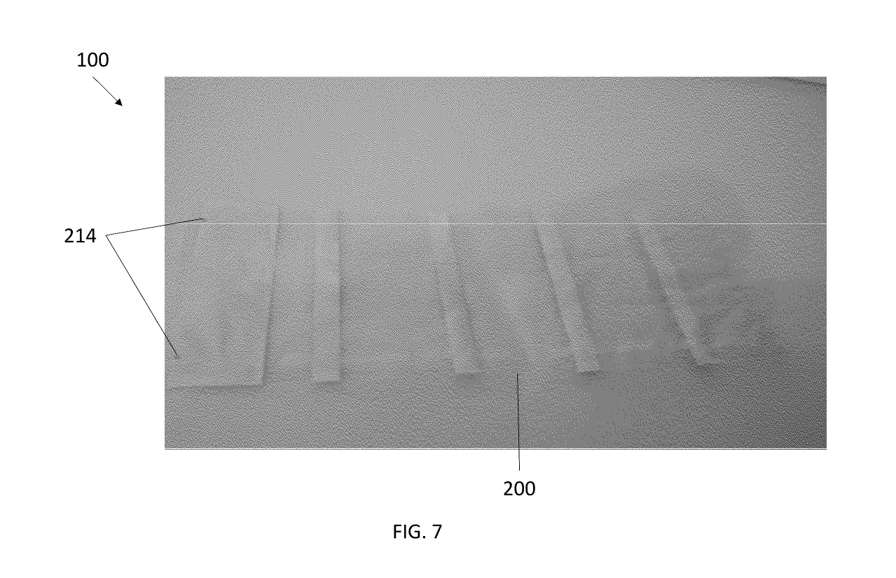

FIG. 7 is an example embodiment of a medication syringe organization apparatus 102 from a back side perspective view in accordance with the present invention.

In the example embodiments of FIGS. 2-13, a single sheet of plastic, rubber, paper, metal or other appropriate material can be provided as a backing 200. In various embodiments, backing 200 can be a single, continuous, uninterrupted, monolithic structure or it can be made of numerous pieces which are coupled together to make up a larger structure. Backing 200 can be free-standing or supported from above, below, sides, or combinations thereof by hooks, legs, arms, tape and others and may include structures such as holes 214. Backing 200 can be rigid, flexible, foldable, stackable, or others. Backing 200 can be a flat sheet. Backing 200 can be cylindrical, box shaped, pentagonal, octagonal, or other shape or structure. Backing 200 can be supported such that it is rotatable about a central axis, twistable, turnable, a combination of one or more, or otherwise manipulable. Backing 200 can be clear, colored or combinations thereof according to particular medical procedures it is associated with, such that an appropriate backing version can be selected quickly and accurately in an emergency or otherwise hectic environment. Different sections of a single backing 200 can be colored in different fashions in order to differentiate from other portions of a single backing.

Numerous pockets 300 can be provided for groups of medication syringes or medication vials for intended for use with specific syringes. Pockets 300 can be the same material as backing 200 (examples provided previously herein) or different materials in various embodiments and can have various dimensions. Pockets 300 can be homogeneous or heterogeneous in size, material, opaqueness, organization or other qualities and attributes in various embodiments. In some embodiments pockets 300 can be further divided and subdivided into size specific compartments for holding a single syringe or other specific numbers of syringes which are related to a particular portion of a related medical procedure or which are holding a particular type of medication. For example, one or more pockets 300 can be designated as a group such as pressors, and individual slots in the one or more pockets can be provided for individual medications within the pressor group such as epinephrine, norepinephrine, phenylephrine, ephedrine, or others. Additional pockets 300 can be used for storage of other medical apparatuses such as tissues, swabs, scalpels, anesthesia equipment, and various other medical devices and items.

In some embodiments pockets 300 can be closed with flaps, fasteners, buttons, snaps, Velcro, or others. Pockets 300 can also be sealed with hook and loop fasteners, adhesives or others. In some embodiments pockets 300 can be removable, attachable or otherwise adjustable. Pockets 300 can be located on one or multiple sides of the system or apparatus 100 in various embodiments. Pockets 300 can be opaque or translucent. Pockets 300 can be connected with adjacent or nonadjacent pockets such that they are grouped together. For instance, two adjacent pockets can contain medication that should be administered simultaneously or in succession. As such, pockets 300 can be linked such that opening or closing one pocket opens or closes a second pocket or otherwise provides access to opening or closing a second or additional pocket. Pockets 300 can be coupled to backing 200 using appropriate means such as stitching, adhesives or others. Pockets 300 can be individual structures or backing 200 can comprise a portion of an individual pocket's structure. In some embodiments pockets 300 can be used in addition to or as an alternative to boxes, bags, slots or other structures. These structures can be a variety of materials including plastic, paper and many others, as appropriate. Pockets 300 can include various interior and exterior shapes and structures which can be rigid, semi-rigid, or flexible. Examples include cylindrical shapes, plastic bag shapes and various others. Pockets 300 can include open top areas for receiving syringes.

Some pockets 300 can be separated such that a first side can hold medication syringes containing medicine to be administered while a second side can hold empty, pre-injection or post-injection medication syringes. In some embodiments a first side of the system or apparatus 100 can hold for pre-injection syringes while a second side can hold post-injection syringes. Pockets 300 can be organized in various fashions as appropriate, for instance in rows 302 and columns 304. In some embodiments, multiple systems and apparatuses 100 can be used together. For instance, a first system 100 may be filled pre-medical procedure with syringes full of medications while a second system 100 can be used for empty syringes, post-administration.

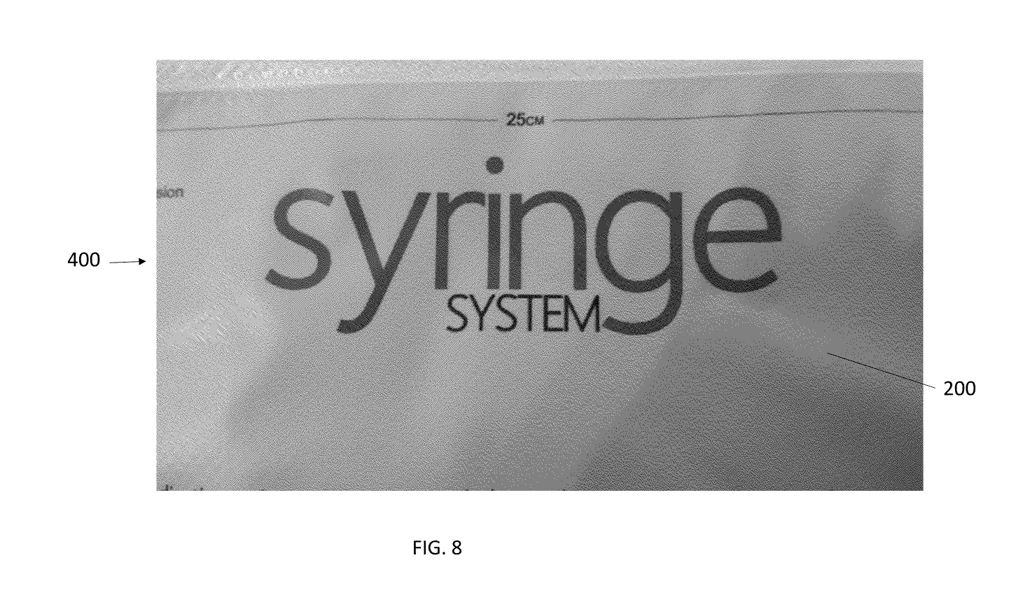

FIG. 8 is an example embodiment of a medication syringe organization apparatus header area 400 in accordance with the present invention.

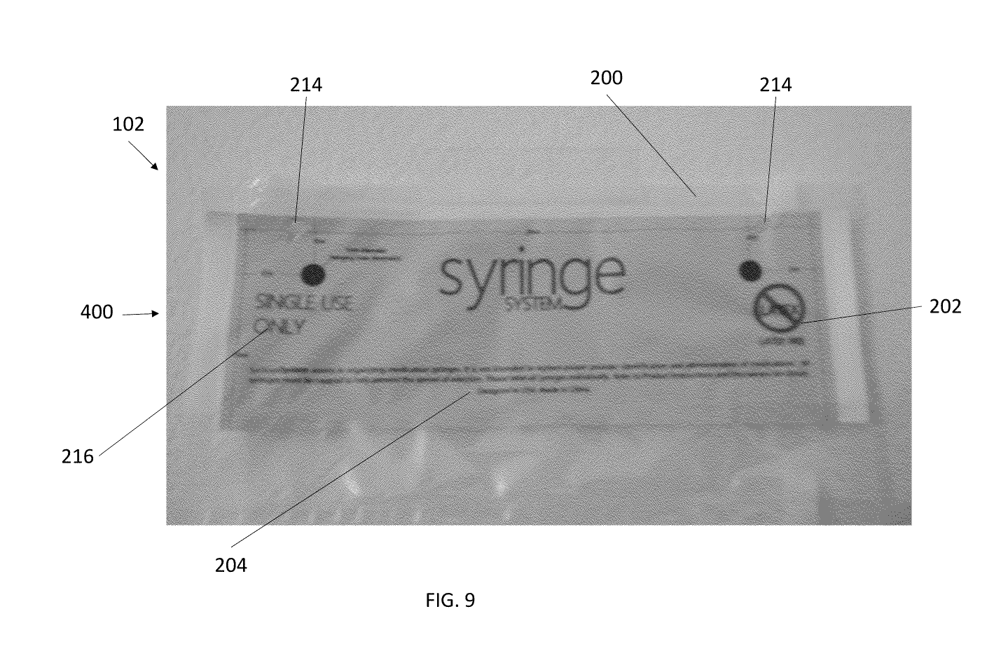

FIG. 9 is an example embodiment of a medication syringe organization apparatus header area 400 in accordance with the present invention.

In the embodiment shown in FIG. 9, a latex free label 202 can indicate to personnel that patients need not be worried about latex allergies and use of the syringe organizer 100 in some embodiments. This can be located in a header area 400 along with various other labels.

Other labels 206 can be printed on backing 200 and pockets 300 in various embodiments as appropriate. A warning label 204 is shown in the example embodiment which reads: “Syringe system assists in organizing medication syringes. It is not intended to replace proper provider identification and administration of medications. All syringes must be capped to help prevent the spread of infection. Please label all syringes individually. Refer to Product Instructions and Disclaimers for details.” Additional warnings can be placed or printed on backing 200 and pockets 300 as appropriate. For example, one pocket 300 can have a warning which advises an administrator not to administer the pocket's contents before administering the contents of another pocket 300. One pocket 300 can also provide an area on the pocket for the medication provider to write in the dosage and/or concentration of the medication to be administered. In some embodiments labels can be removable, changeable or otherwise movable.

Other labeling can be useful in some embodiments. For example, metric or other size, length or distance labels can be included on pockets 300 and backing 200 to provide simple and immediate recognition of particular syringes. A one-inch diameter syringe can be placed in a location where its size is indicated. This can be adjacent to a three-quarters or an inch diameter syringe which is also located where its size is indicated. This provides instant recognition of whether the syringes are housed in their appropriate locations based on whether the markings match up. Likewise, height indicators can indicate the height of a syringe and, in some embodiments, the height of medication in a syringe.

The systems, methods, and apparatuses 100 described herein can provide instant recognition in the form of color coordination. Alternatively or additionally, shapes can be used to provide instant recognition such as triangles, squares, pentagons, stars, and other polygons. Patterns 210 can also be used including stripes, hash marks, dashes, dots, zig-zags, and many others. Numbering can also be used. As an additional measure, in many embodiments a written label with the full name and dose of the medication contained in a syringe located therein can be included. This feature can provide a backup in the event an individual forgets or wishes to double check a particular medication in addition to the instant recognition. The systems, methods and apparatuses 100 herein allow providers to organize syringes filled with medications or syringes intended to be used with certain medications or medication vials intended to be used with certain syringes to be placed in appropriately labeled groups. The systems, methods and apparatuses 100 can also allow providers to organize syringes filled with medications or syringes intended to be used with certain medications or medication vials intended to be used with certain syringes to be placed in appropriately specific medication labeled individual locations.

In the example embodiment, a combination of color 208 and pattern 210 coding is shown as well as clear labeling and inclusion of text 212. Examples of text 212 and associated colors 208 and patterns 210 will now be described with regard to the example embodiment.

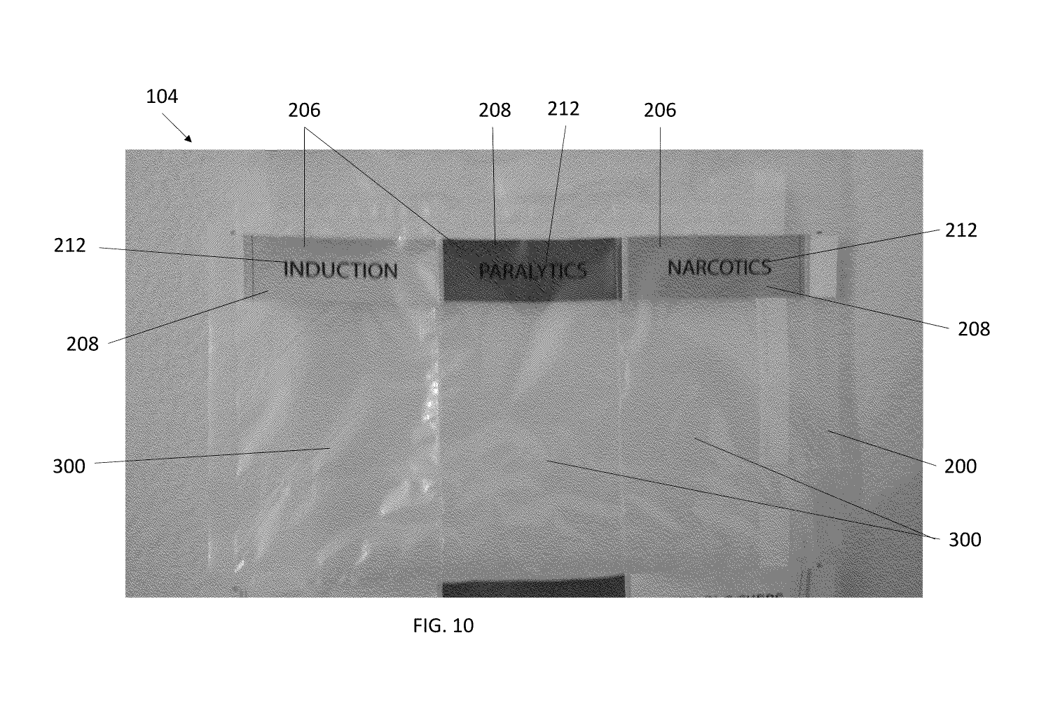

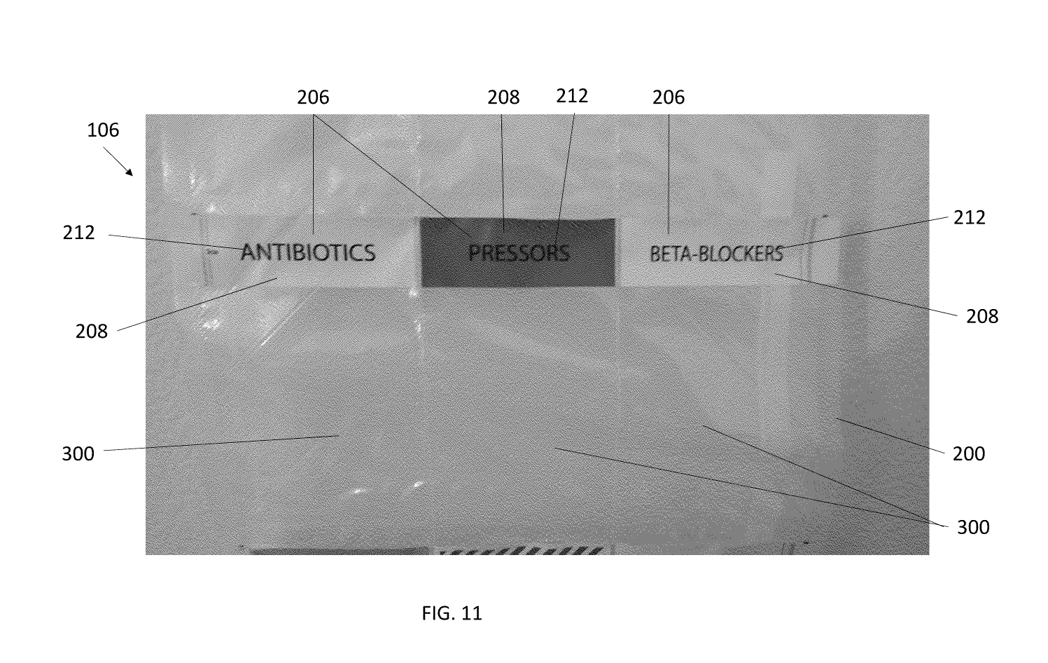

FIG. 10 is an example embodiment of a medication syringe organization apparatus pocket area first row 104 in accordance with the present invention. In the first row 104, a label 206 of an “Induction” 212 pocket 300 can be colored 208 solid yellow, a label 206 of a “Paralytics” 212 pocket 300 can be colored 208 solid red and a label 206 of a “Narcotics” 212 pocket 300 can be colored 208 solid blue.

FIG. 11 is an example embodiment of a medication syringe organization apparatus pocket area second row 106 in accordance with the present invention. In the second row 106, a label 206 of an “Antibiotics” 212 pocket 300 can be colored solid white 208, a label 206 of a “Pressors” 212 pocket 300 can be colored solid purple 208 and a label 206 of a “Beta-Blockers” 212 pocket 300 can be colored solid white 208.

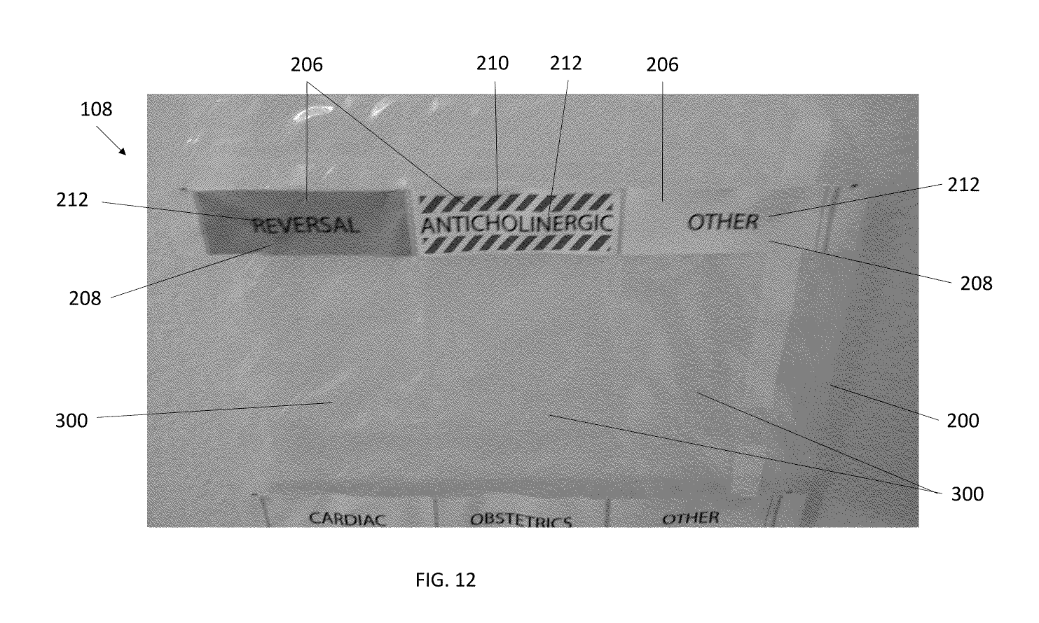

FIG. 12 is an example embodiment of a medication syringe organization apparatus pocket area third row 108 in accordance with the present invention. In the third row 108, a label 206 of a “Reversal” 212 pocket 300 can be colored solid lime green 208, a label 206 of an “Anticholinergic” 212 pocket 300 can be colored white 208 with red hash marks or diagonal stripes pattern 210 and a label 206 of a first “Other” 212 pocket 300 can be colored solid white 208.

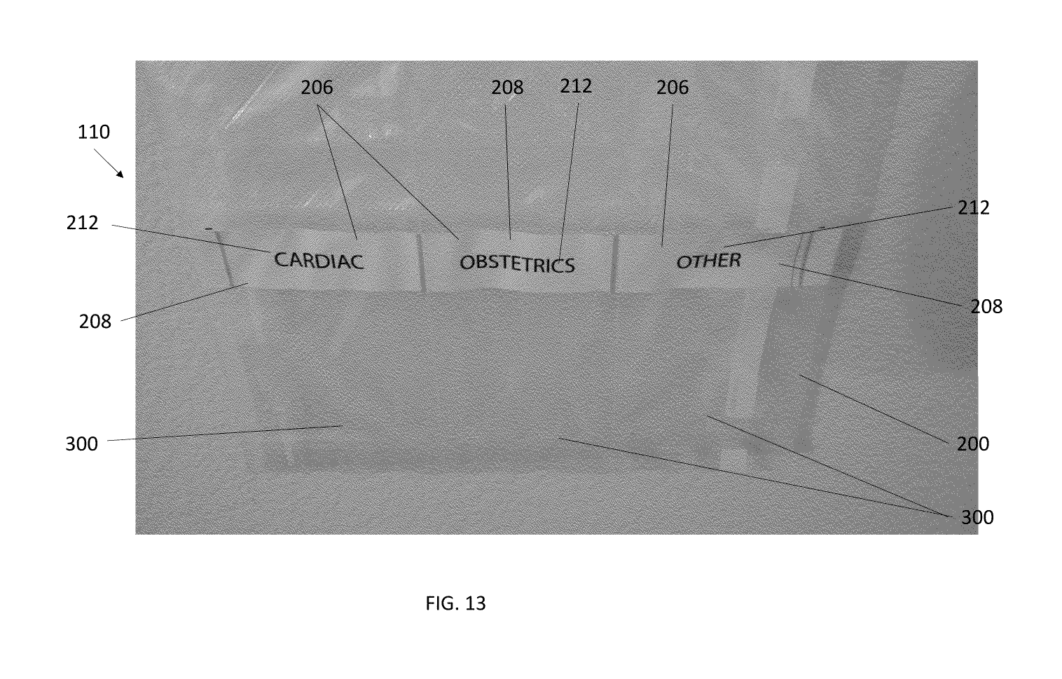

FIG. 13 is an example embodiment of a medication syringe organization apparatus pocket area fourth row 110 in accordance with the present invention. In the fourth row 110, a label 206 of a “Cardiac” 212 pocket 300 can be colored solid white 208, a label 206 of an “Obstetrics” 212 pocket 300 can be colored solid white 208 and a label 206 of a second “Other” 212 pocket 300 can be colored solid white 208.

In various embodiments, related pockets can have identical colors or different shades of the same color as appropriate. Similarly, administration can be color coordinated according to procedure, such as using the acronym ROYGBIV (Red, Orange, Yellow, Green, Blue, Indigo, and Violet) where each color can be used to indicate the order of the procedure. In some embodiments, health care providers can update, change or reclassify portions of the system as appropriate or necessary with minimal effort. As an example, in the embodiment shown, one of the “Other” labeled pockets can be updated to “Antiemetics” by writing over with pen, by attaching a label with an adhesive or other appropriate means.

Administration of medication can also be unrelated to color but still organized in particular fashions using these apparatuses, systems and methods. For example, in a cardiac surgery, medication A can be administered first, then medication B, then medication C, then medication D, and so on. The syringe system can organize the syringes in order of planned administration. Some embodiments may be organized in a left to right, top to bottom fashion but others are also contemplated. Medications syringes can also be organized in terms of classes based on particular operating and procedural suites.

In an example embodiment, syringes containing medication can be color and pattern coordinated with the pocket indicating color to ensure accuracy. As such a syringe containing a “Narcotic” medication can have the same blue coloring as a pocket with blue coloring.

In some embodiments the system or apparatus 100 can be a non-sterile, single use (as indicated by label 216), disposable, organization system for medication syringes for surgical and procedural rooms/suites. In other embodiments, the system can be sterile or reusable as appropriate. In many embodiments the system or apparatus 100 can provide organization for anesthesiologists, associated anesthesia providers, and other medical and dental staff using medication syringes to administer to patients.

Various embodiments of the system, apparatus and methods described herein can be procedure specific, patient specific or operating room specific. In a procedure specific embodiment, the system, apparatus and methods can be oriented for the requirements of the specific procedure, such as cardiac surgery, liver surgery, obstetric surgery or others. For example, a first procedure may require administration of ten different medications while a second procedure may require administration of six medications, some of which can be the same medication administered at different steps of the procedure. Similarly, a medical procedure for one patient may differ from a second patient due to allergies, gender, weight, or other factors and therefore different embodiments can be set up or administered differently. Similarly, an operating room in a large hospital can be set up differently from one in a smaller clinic. In such instances space considerations can be different and various embodiments can account for these differences.

In some embodiments, additional structures can be included. In an example embodiment, a plurality of blocking sheets can be folded such that individual rows 302 or columns 304 of pockets 300 are blocked from normal view. This can prevent inadvertent removal or replacement of syringes from pockets at incorrect steps in a procedure. Blocking sheets can be attached at particular spots using buttons, hooks, adhesives, clasps or other appropriate attachment means.

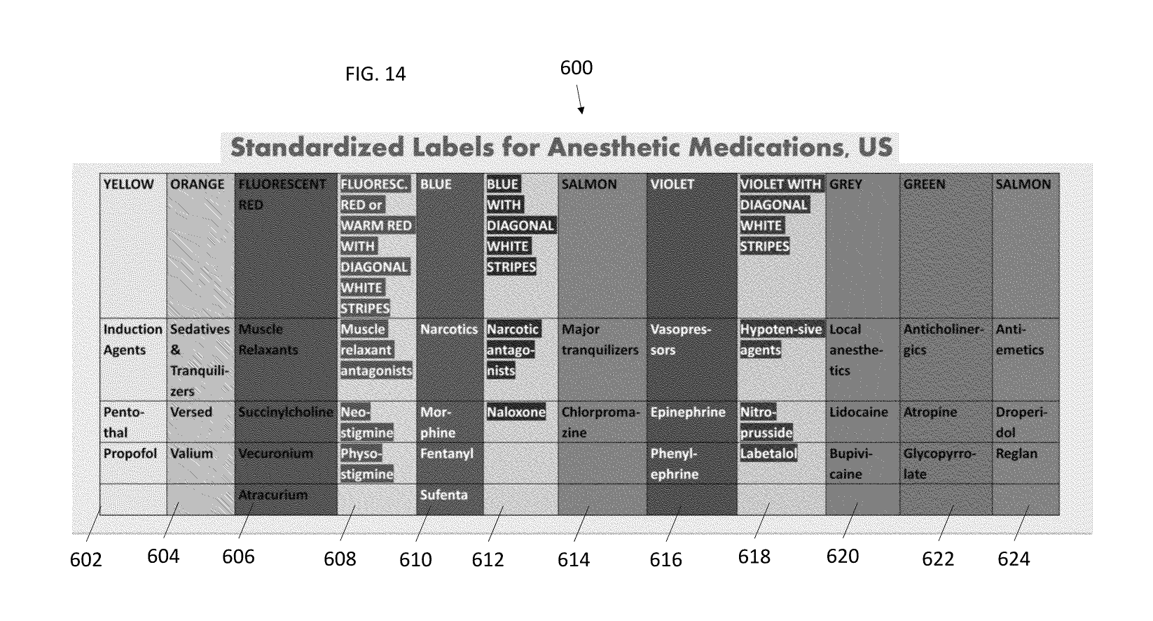

FIG. 14 is an example embodiment of a standardized label coloring chart 600 for anesthetic medications in the United States in accordance with the present invention.

In the example embodiment, chart 600 includes various columns 602-624 which will be described in detail. Column 602 corresponds to a yellow color for induction agents such as Pentothal and Propofol. Column 604 corresponds to an orange color for sedatives and tranquilizers such as Versed and Valium. Column 606 corresponds to a fluorescent red color for muscle relaxants such as Succinylcholine, Vecuronium and Atracurium. Column 608 corresponds to a fluorescent red or warm red with diagonal white stripes color for muscle relaxant antagonists such as Neostigmine and Physostigmine. Column 610 corresponds to a blue color for narcotics such as Morphine, Fentanyl and Sufenta. Column 612 corresponds to a blue with diagonal white stripes color for narcotic antagonists such as Naloxone. Column 614 corresponds to a salmon color for major tranquilizers such as Chlorpromazine. Column 616 corresponds to a violet color for vasopressors such as Epinephrine and Phenylephrine. Column 618 corresponds to a violet with diagonal white stripes color for hypotensive agents such as Nitroprusside and Labetalol. Column 620 corresponds to a gray color for local anesthetics such as Lidocaine and Bupivicaine. Column 622 corresponds to a green color for anticholinergics such as Atropine and Glycopyrrolate. Column 624 corresponds to a salmon color for antiemetics such as Droperidol and Reglan.

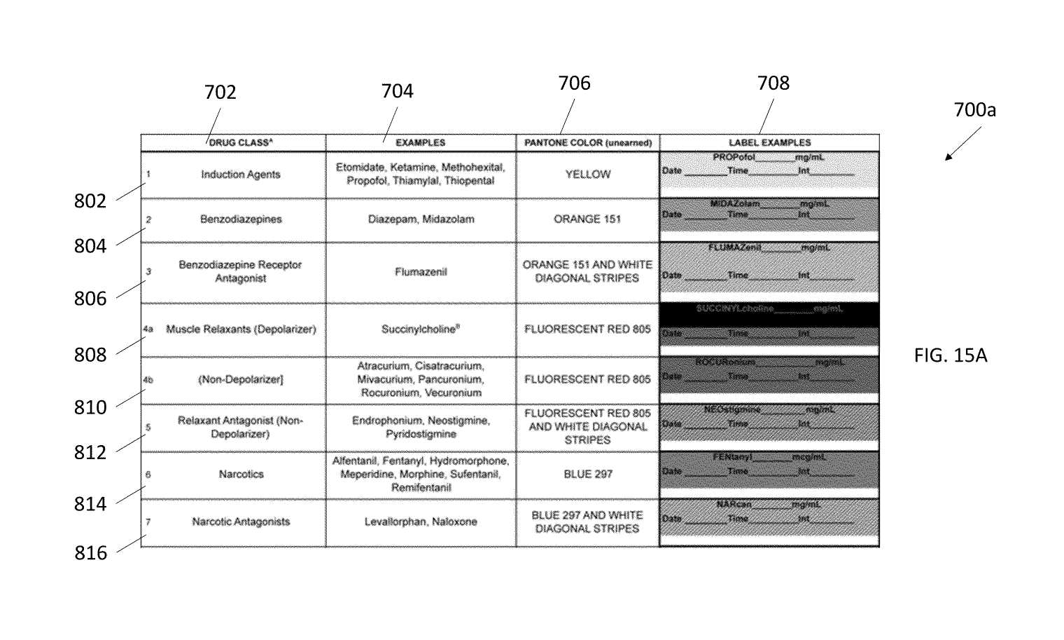

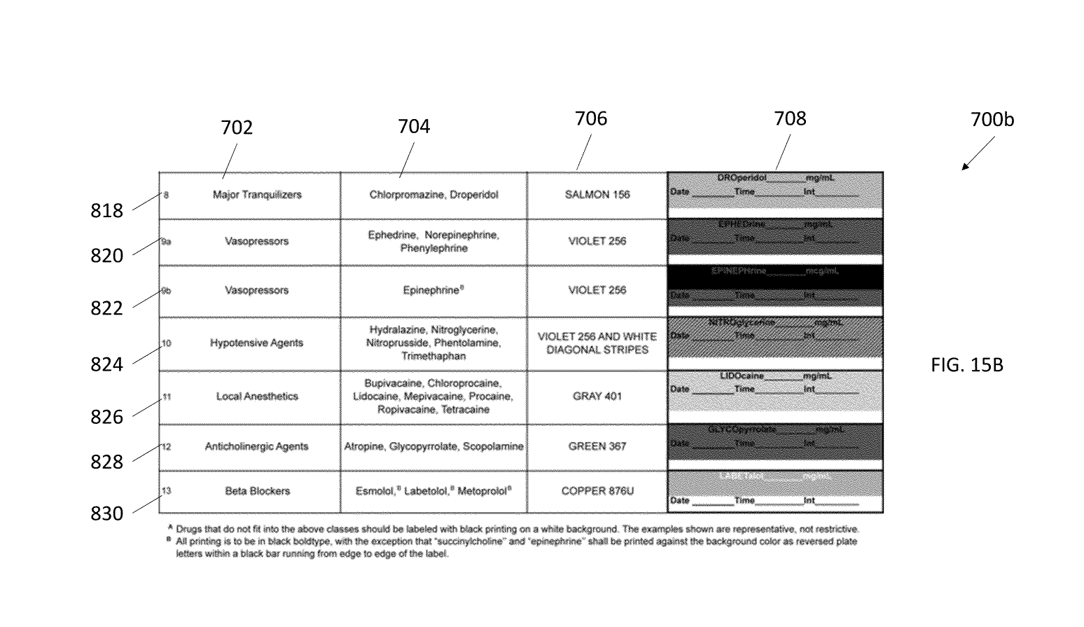

FIGS. 15A-15B show an example embodiment of a first and second section 700a-700b respectively of a classification chart created and published by the American Society for Testing and Materials (ASTM) showing a standard for user-applied labels in an operating room (OR) as described in ASTM D4774-06 “Standard Specification for User Applied Drug Labels in Anesthesiology.”

As shown in the example embodiment, column 702 shows a drug class, 704 shows examples, 706 shows a corresponding Pantone Color and column 708 shows an example label. Example labels can include a drug name, date, time, amount, and administrator initials.

Turning to FIG. 15A, Row 802 corresponds to a yellow color for Induction Agents such as Etomidate, Ketamine, Methohexital, Propofol, Thiamyfall and Thiopental. Row 804 corresponds to an orange color for Benzodiazepines such as Diazepam and Midazolam. Row 806 corresponds to an Orange and White Diagonal Stripes color for Benzodiazepine Receptor Antagonist such as Flumazenil. Row 808 corresponds to a Fluorescent Red color for Muscle Relaxants (depolarizer) such as Succinylcholine. Row 810 corresponds to a Fluorescent Red color for Muscle Relaxants (non-depolarizer) such as Atracurium, Cisatracurium, Mivacurium, Pancuronium, Rocuronium and Vecuronium. Row 812 corresponds to a Fluorescent Red and White Diagonal Strips color for Relaxant Antagonist (non-depolarizer also known as Reversal) such as Endrophonium, Neostigmine and Pyridostigmine. Row 814 corresponds to a Blue color for Narcotics such as Alfentanil, Fentanyl, Hydromorphone, Meperidine, Morphine, Sufentanil and Remifentanil. Row 816 corresponds to a Blue and White Diagonal Stripes color for Narcotic Antagonists such as Levallorphan and Naloxone.

Turning to FIG. 15B, Row 818 corresponds to a Salmon color for Major Tranquilizers such as Chlorpomazine and Droperidol. Rows 820 and 822 correspond to a Violet color for Vasopressors such as Ephedrine, Norepinephrine, Phenylephrine and Epinephrine. Row 824 corresponds to a Violet and White Diagonal Stripes color for Hypotensive Agents such as Hydralazine, Nitroglycerine, Nitroprusside, Phentolamine and Trimethaphan. Row 826 corresponds to a Gray color for Local Anesthetics such as Bupivacaine, Chloroprocaine, Lidocaine, Mepivacaine, Procaine, Ropivacaine and Tetracaine. Row 828 corresponds to a Green color for Anticholinergic Agents such as Atropine, Glycopyrrolate and Scopolamine. Row 830 corresponds to a Copper color for Beta Blockers such as Esmolol, Labetolol and Metoprolol.

In some embodiments, for various drugs that do not fit into the above classes for FIGS. 15A-15B, they can be labeled or indicated with labels having black printing on a white background. While the examples shown in the example embodiment are representative they are not restrictive of all example drugs in each class, nor all classes possible. Printing can be done in black bold typeface with some exceptions. In some embodiments, Succinylcholine and Epinephrine can be printed against a background color as reversed plate letters within a black bar running from edge to edge on a label.

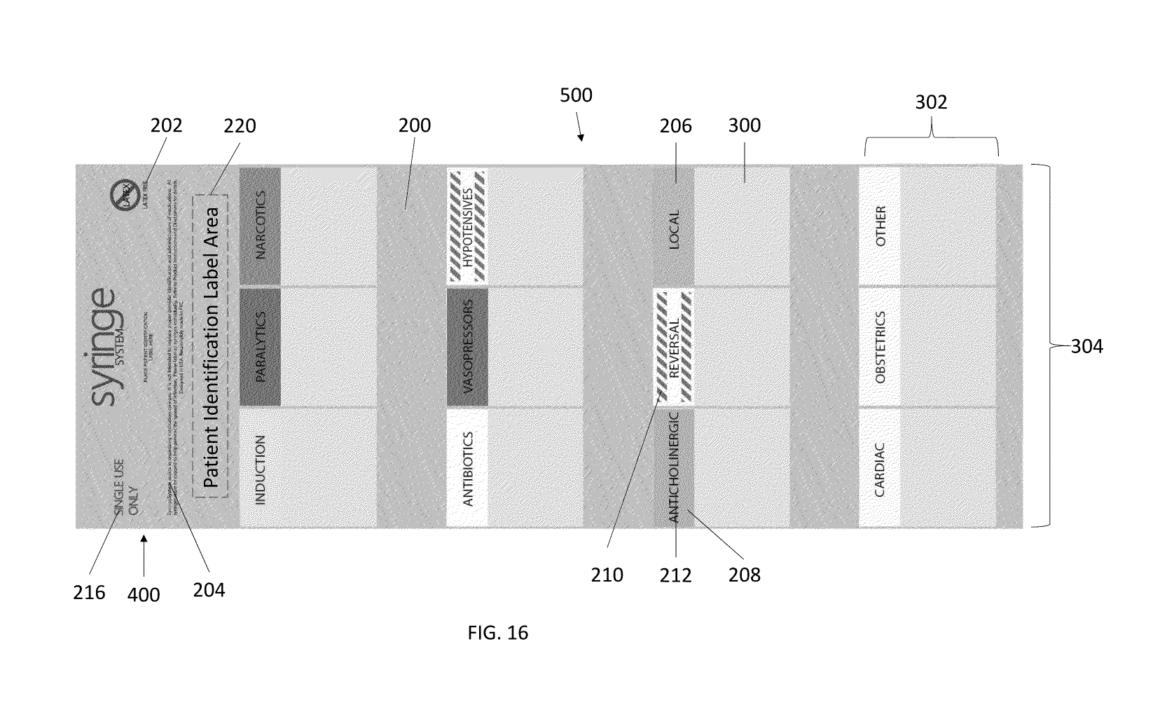

FIG. 16 is an example embodiment of a medication syringe organization apparatus diagram 500 in accordance with the present invention. In the example embodiment header area 400 includes a Patient Identification Label Area 220 which can include one or more of a patient name, description of patient procedure, patient number or other identifying information. In some embodiments Area 220 can be a series of descriptions with lines next to them for a healthcare professional to write information directly on backing 200 in the appropriate location using a marker, pen or other writing utensil. In other embodiments this are can include components useful in affixing or coupling patient identification labels to backing 200 including and not limited to one or more of clips, adhesive strips, felt backing, Velcro type fastener backing and others as appropriate. Area 220 can serve to decrease a risk of using one or more of apparatus 500 and its associated syringes on multiple patients and can help in organization of multiple apparatuses 500.

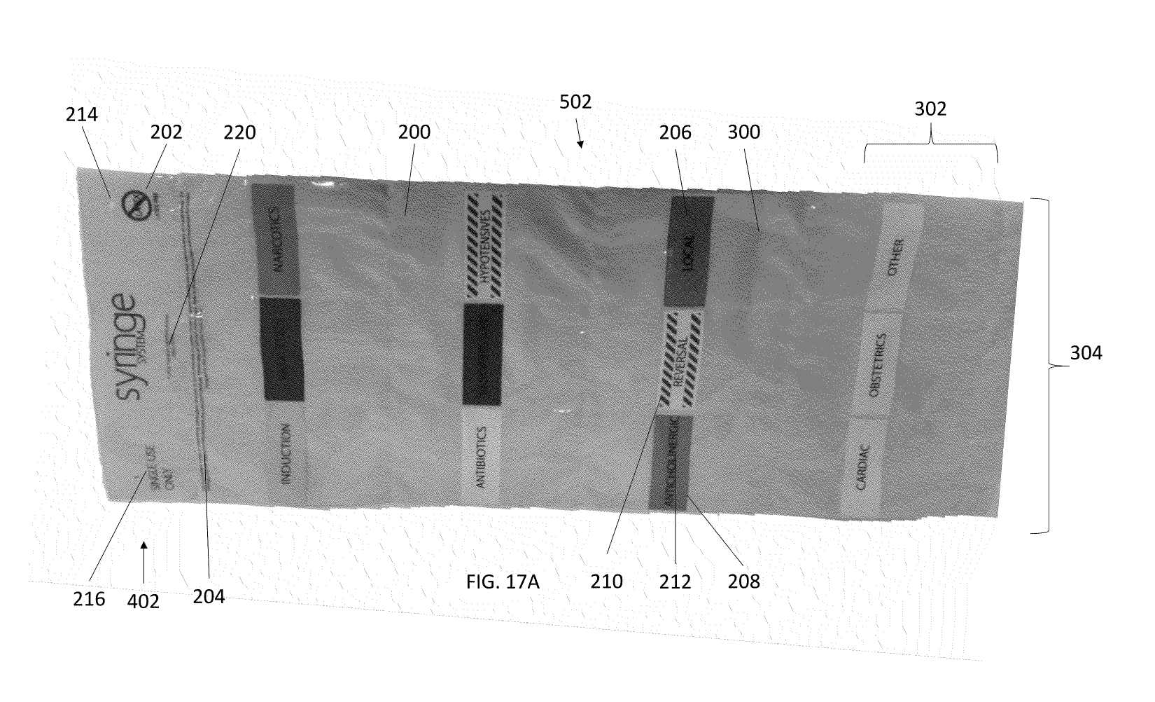

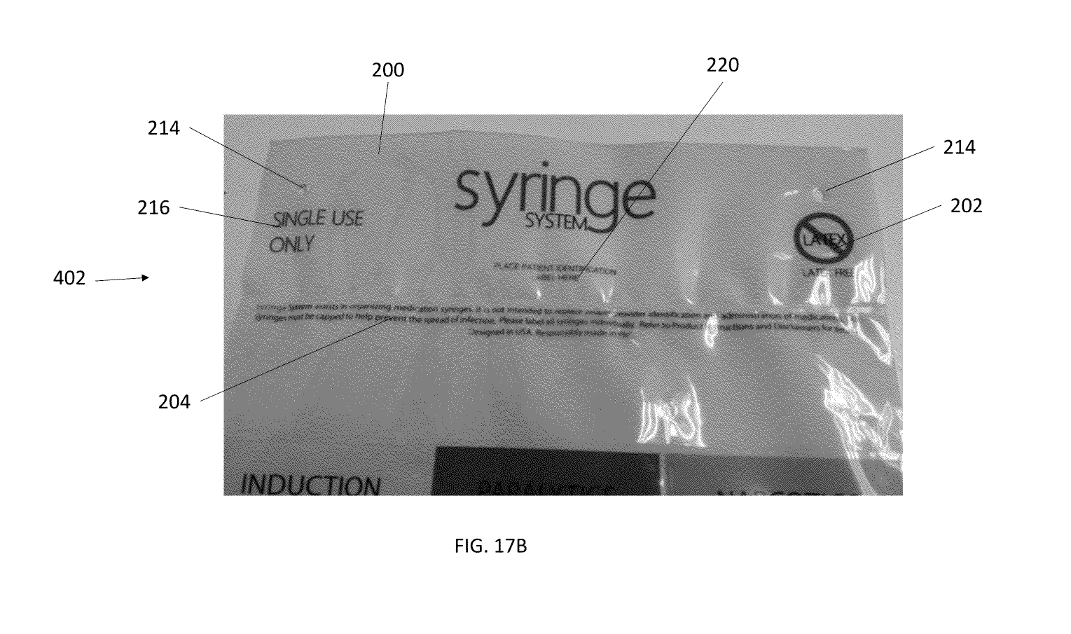

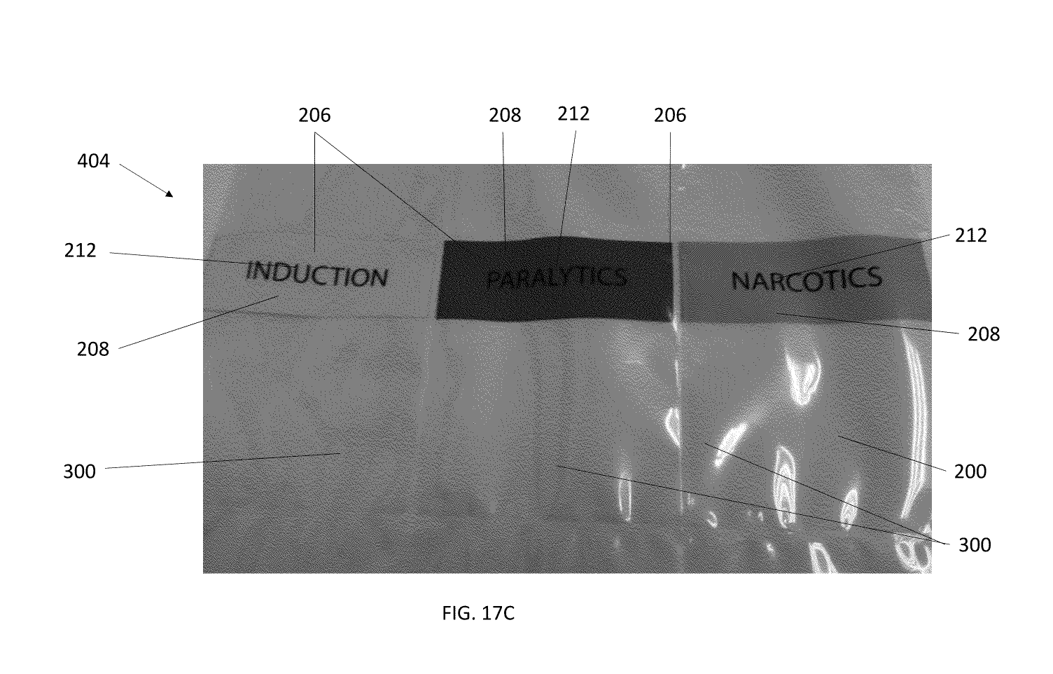

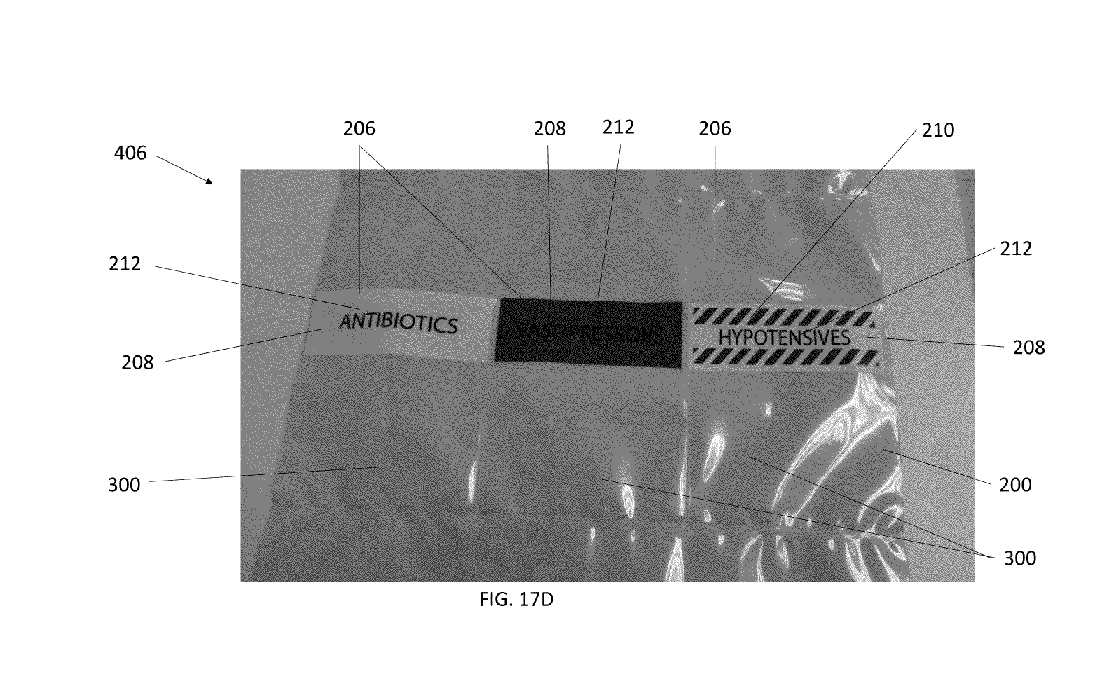

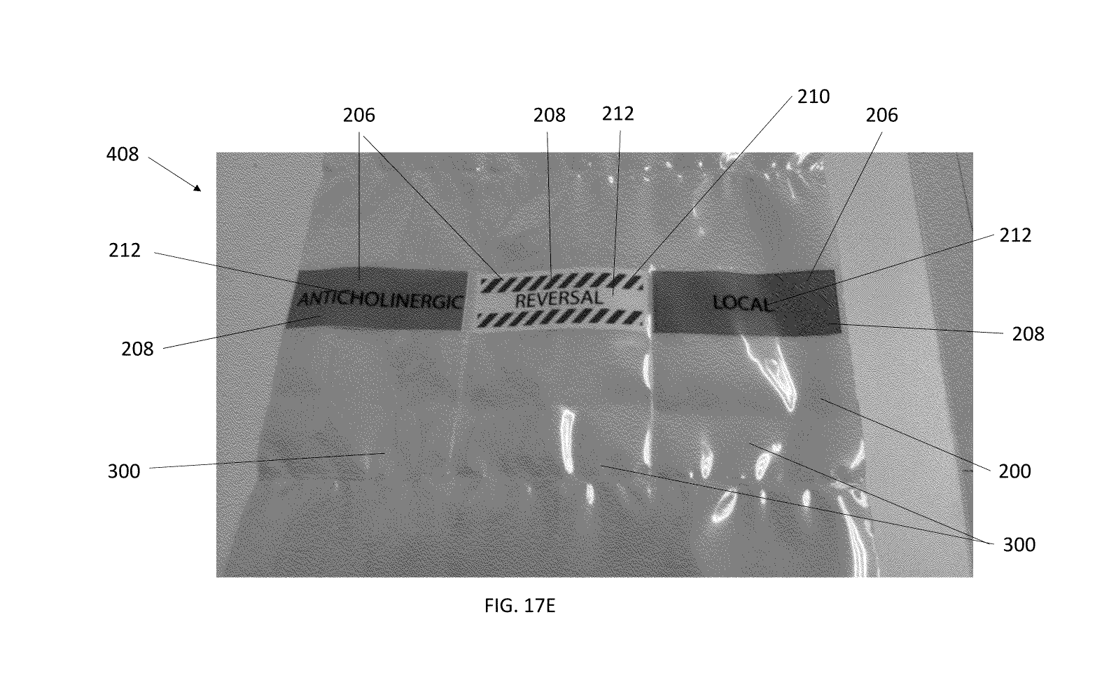

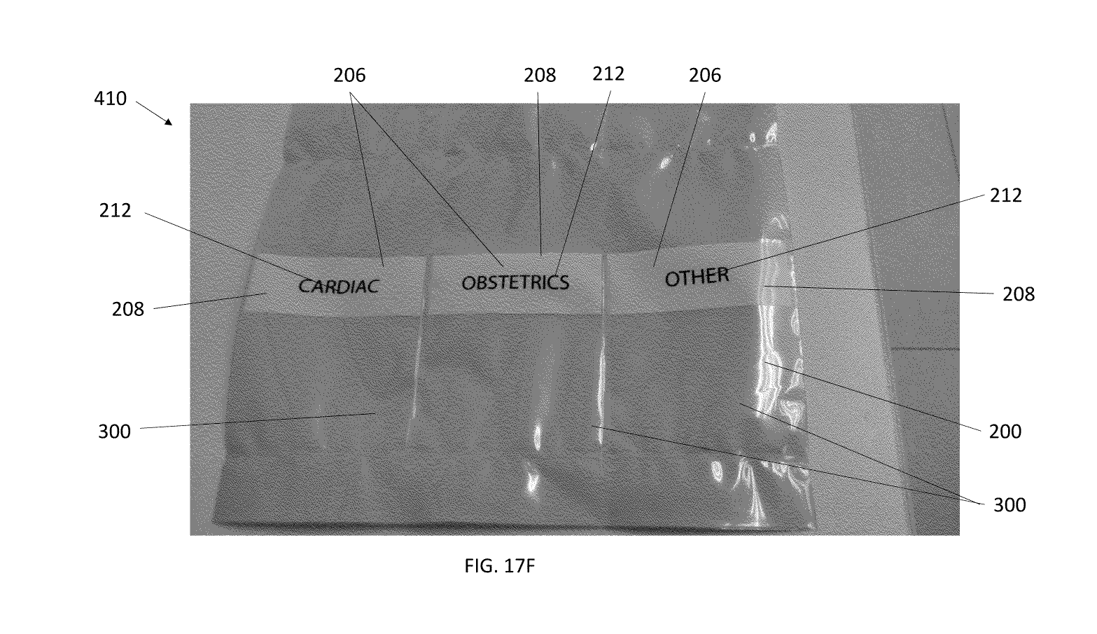

Examples of text 212 and associated colors 208 and patterns 210 are similar in the example embodiment of FIGS. 16 and 17A-F as in FIGS. 2-13.

FIG. 17A is an example embodiment of a medication syringe organization apparatus 502 from a front side perspective view in accordance with the present invention.

Some differences between FIGS. 16 and 17A-F as in FIGS. 2-13 are as follows:

FIG. 17B is an example embodiment of a medication syringe organization apparatus 502 header area 402 in accordance with the present invention. In FIGS. 16 and 17A-17B, header area 402 includes patient identification label area 220.

FIG. 17C is an example embodiment of a medication syringe organization apparatus 502 pocket area first row 404 in accordance with the present invention.

FIG. 17D is an example embodiment of a medication syringe organization apparatus 502 pocket area second row 406 in accordance with the present invention. In second row 406, a label 206 of a “VASOPRESSORS” 212 pocket 300 can be colored pink or purple 208, while a label 206 of a “HYPOTENSIVES” 212 pocket 300 can be colored violet 208 with diagonal stripes pattern 210 to designate hypotensive medications.

FIG. 17E is an example embodiment of a medication syringe organization apparatus 502 pocket area third row 408 in accordance with the present invention. In third row 408, a label 206 of a “REVERSAL” 212 pocket 300 can be colored white 208 with red hash marks or diagonal stripes pattern 210 to designate a muscle relaxant reversal agent or muscle relaxant antagonist category, a label 206 of an “Anticholinergic” 212 pocket 300 can be colored solid lime green 208 and a label 206 of a “LOCAL” 212 pocket 300 can be colored gray 208 to designate local anesthetics.

FIG. 17F is an example embodiment of a medication syringe organization apparatus 502 pocket area fourth row 410 in accordance with the present invention.

In some embodiments a tray or other organization area on a flat surface can be used to implement the principles described herein. For example, color coded stickers, with or without classification labels in various embodiments, can provide a location for differentiation in medication syringe organization. In some embodiments trays may include separate bins or other physical separation structures.

As used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

In many instances entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic) intervening entities) and the indirect coupling of two entities (with one or more non-negligible intervening entities). Where entities are shown as being directly coupled together, or described as coupled together without description of any intervening entity, it should be understood that those entities can be indirectly coupled together as well unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

What is claimed is:

1. A system of syringe organization comprising:
a backing;
a plurality of pockets coupled to the backing and operable to maintain syringe organization; and
a coordination scheme indicating the contents of individual pockets.

2. The system of syringe organization of claim 1, wherein the backing is a monolithic structure.

3. The system of syringe organization of claim 1, wherein the pockets further comprise a translucent material and the backing comprises a wall of a pocket.

4. The system of syringe organization of claim 1, wherein the coordination scheme includes colors.

5. The system of syringe organization of claim 1, wherein the coordination scheme includes text.

6. The system of syringe organization of claim 5, wherein the coordination scheme colors can be used to group syringes based on the properties of the medications they contain.

7. The system of syringe organization of claim 1, wherein the coordination scheme includes patterns.

8. The system of syringe organization of claim 1, wherein the coordination scheme includes a combination of at least two of color, text and patterns.

9. The system of syringe organization of claim 1, wherein the coordination scheme includes organization of pockets into rows and columns.

10. The system of syringe organization of claim 1, wherein the backing includes one or more holes with which the backing can be supported by hooks or otherwise.

11. The system of syringe organization of claim 1, wherein the at least one pocket is closable using a flap.

12. The system of syringe organization of claim 11, wherein the flap can be secured using a button or clip.

13. The system of syringe organization of claim 1, wherein the backing comprises rubber, plastic, paper, or metal.

14. The system of syringe organization of claim 1, wherein the pockets are homogeneous in size.

15. The system of syringe organization of claim 1, wherein the pockets are heterogeneous in size.

16. The system of syringe organization of claim 1, wherein the coordination scheme includes organization of pockets based on a particular medical procedure.

17. The system of syringe organization of claim 1, wherein the coordination scheme includes organization of pockets based on an order of medication application during a particular medical procedure.

18. The system of syringe organization of claim 1, wherein at least one pocket is used for syringe disposal after administration of a medication.

19. The system of syringe organization of claim 1, wherein at least one pocket is used for housing medical implements other than syringes.

20. The system of syringe organization of claim 1, wherein at least one pocket is removable from the backing.
